(19) United States
(12) Patent Application Publication
(10) Pub. No.: US 2020/0222508 A1
(43) Pub. Date: Jul. 16, 2020

(54) GLUCAGON-LIKE PEPTIDE 1 (GLP-1) RECEPTOR AGONIST COMPOSITIONS

(71) Applicant: Arecor Limited, Little Chesterford, Saffron Walden (GB)

(72) Inventors: Jan JEZEK, Little Chesterford, Saffron Walden (GB); David GERRING, Little Chesterford, Saffron Walden (GB); Sarah HOWELL, Little Chesterford, Saffron Walden (GB)

(21) Appl. No.: 16/471,596

(22) PCT Filed: Dec. 22, 2017
(86) PCT No.: PCT/GB2017/053879

§ 371 Date: Jun. 20, 2019

(30) Foreign Application Priority Data
Dec. 22, 2016 (GB) 1621987.5

(51) Int. Cl. A61K 38/26; A61K 9/08; A61K 47/02; A61K 47/34; A61K 47/18; A61K 9/00

(52) U.S. Cl. CPC A61K 38/26; A61K 9/08; A61K 9/0019; A61K 47/34; A61K 47/18; A61K 47/02

(57) ABSTRACT

There is provided, inter alia, an aqueous solution composition comprising a GLP-1 receptor agonist as an active ingredient and multivalent anions having a charge of at least minus 2 as stabilising agent, wherein the total concentration of multivalent anions in the composition having a charge of at least minus 2 is at least 15 mM.

This invention relates to aqueous solution compositions of glucagon-like peptide-1 (GLP-1) receptor agonists and their use in therapy.

### BACKGROUND

Glucagon-like peptide-1 (GLP-1) is an incretin derived from the transcription product of the proglucagon gene. The biologically active forms of GLP-1 are: GLP-1(7-37) and GLP-1(7-36)NH2, fragments resulting from selective cleavage of the proglucagon molecule. GLP-1 (in its various biologically active forms) is an agonist of the glucagon-like peptide 1 receptor (GLP-1 receptor) and has potent insulinotropic effects in stimulating insulin secretion and inhibiting glucagon secretion. These glucose-lowering effects make GLP-1 an important molecule in the treatment of diabetes. One of the potential advantages of the use of GLP-1 over insulin therapy is that insulin production is only stimulated when blood glucose levels are elevated, thereby reducing the risk of hypoglycaemia.

GLP-1 is not used as a therapeutic agent directly because once in circulation it has a half-life of less than 2 minutes, due to rapid degradation by the enzyme dipeptidyl peptidase-4 (DPP4). However, a number of alternative GLP-1 receptor agonists have been developed with an increased circulating half-life.

GLP-1 receptor agonists approved by regulatory authorities include:
albiglutide (marketed under the tradenames Eperzan/Tanzeum)
dulaglutide (marketed under the tradename Trulicity)
exenatide (marketed under the tradenames Byetta/Bydureon)
liraglutide (marketed under the tradenames Victoza/Saxenda)
lixisenatide (marketed under the tradename Lyxumia/Adlyxin)

Tanzeum Pen for injection (for subcutaneous use) contains 67 mg lyophilized albiglutide and 0.65 mL water for injection designed to deliver a dose of 50 mg in a volume of 0.5 mL after reconstitution. Inactive ingredients include 153 mM mannitol, 0.01% (w/w) polysorbate 80, 10 mM sodium phosphate, and 117 mM trehalose dihydrate.

Trulicity is a clear, colourless, sterile solution. Each 0.5 mL of Trulicity solution contains 0.75 mg or 1.5 mg of dulaglutide. Each single-dose pen or prefilled syringe contains 0.5 mL of solution and the following excipients: citric acid anhydrous (0.07 mg), mannitol (23.2 mg), polysorbate 80 (0.10 mg) and trisodium citrate dihydrate (1.37 mg) in water for injection.

Byetta (exenatide injection) is supplied for subcutaneous injection as a sterile, preserved isotonic solution in a glass cartridge that has been assembled in a pen-injector. Each 1 mL contains 250 μg synthetic exenatide, 2.2 mg m-cresol as an antimicrobial preservative, mannitol as a tonicity-adjusting agent, and glacial acetic acid and sodium acetate trihydrate in water for injection as a buffering solution at pH 4.5.

Bydureon is a white to off-white powder that is available in a dosage strength of 2 mg exenatide per vial or per pen. Exenatide is incorporated in an extended-release microsphere formulation containing a 50:50 poly(D,L-lactide-co-glycolide) polymer (37.2 mg per dose) along with sucrose (0.8 mg per dose). The powder must be suspended in the diluent prior to injection. The diluent is composed of sodium carboxymethylcellulose (19 mg), polysorbate 20 (0.63 mg), sodium phosphate monobasic monohydrate (0.61 mg), sodium phosphate dibasic heptahydrate (0.51 mg), sodium chloride (4.1 mg) and water for injection.

Victoza is a clear, colourless or almost colourless solution. Each 1 mL of Victoza solution contains 6 mg of liraglutide and the following inactive ingredients: disodium phosphate dihydrate (1.42 mg); propylene glycol (14 mg); phenol (5.5 mg); and water for injection.

Saxenda is a clear, colourless solution. Each 1 mL of Saxenda solution contains 6 mg of liraglutide and the following inactive ingredients: disodium phosphate dihydrate (1.42 mg); propylene glycol (14 mg); phenol (5.5 mg); and water for injection. Each pre-filled pen contains a 3 mL solution of Saxenda equivalent to 18 mg liraglutide (free-base, anhydrous).

Lyxumia/Adlyxin injection is a sterile, clear, colourless aqueous solution for subcutaneous administration supplied in two single-patient use prefilled pens. Each prefilled pen contains 3 mL solution and each mL contains 50 or 100 μg lixisenatide. Inactive ingredients are glycerol 85% (54 mg), sodium acetate trihydrate (10.5 mg), methionine (9.0 mg), m-cresol (8.1 mg), and water for injection.

The GLP-1 receptor agonist products are presented in pre-filled devices (typically pre-filled pens) and are self-administered by the patients. However, in order to maintain the stability of the GLP-1 receptor agonist in the composition during storage and during the in-use period (i.e. the period following the initial use of the pre-filled device during which the pre-filled pen is used repeatedly by the patient until the pen cartridge becomes empty) patients must ensure that the devices are stored and used correctly.

All of the currently marketed GLP-1 receptor agonist products have to be stored at 2-8° C. for their entire shelf-life except the in-use period. During the in-use period, after the initial use of the Victoza/Saxenda (liraglutide) pen, the pen can be stored at temperatures up to 30° C. for up to 30 days. Trulicity (dulaglutide) and Lyxumia/Adlyxin (lixisenatide) pens can be stored at temperatures up to 30° C. for up to 14 days after the initial use. Byetta (exenatide) pen can be stored at temperatures up to 25° C. for up to 30 days. Bydureon (exenatide) vial can be stored at temperatures up to 30° C. for up to 4 weeks prior its use, but must be used immediately after reconstitution. Eperzan/Tanzeum (albiglutide) pens can be stored at temperatures up to 30° C. for up to 4 weeks during the in-use period.

In order to improve patients' convenience and shipment logistics there is a need to improve the stability of compositions of GLP-1 receptor agonists. Desirable compositions would have one or more of the following properties:
the temperature at which the product can be kept during the in-use period is increased, e.g. to 40° C.
the duration of the in-use period is increased, e.g. to 1 month, to 2 months, and preferably to 3 months
the product can be stored at increased temperature, such as controlled room temperature (20-25° C.) for part of the shelf-life, e.g. 3 months, 6 months or the entire shelf-life, whilst maintaining the in-use stability at 30° C.

Thus, an object of the present invention is the provision of an aqueous solution composition of a GLP-1 receptor agonist as active ingredient with improved stability.

### SUMMARY OF THE INVENTION

According to the invention, there is provided an aqueous solution composition comprising a GLP-1 receptor agonist as an active ingredient and multivalent anions having a charge of at least minus 2 as stabilising agent, wherein the total concentration of multivalent anions in the composition having a charge of at least minus 2 is at least 15 mM.

### FIGURES

FIG. 1: Stability of liraglutide in the presence of propylene glycol, mannitol, trehalose or sodium chloride as additives, assessed by RP-HPLC following storage at 30° C. for 9 and 14 weeks (see Table 1 of Example 1).

FIG. 2: Stability of liraglutide in the presence of propylene glycol, mannitol or histidine (with and without mannitol) as additives, assessed by RP-HPLC following storage at 30° C. for 9 and 14 weeks (see Table 1 of Example 1).

FIG. 3: Stability of liraglutide in the presence of citrate anions (with and without mannitol), assessed by RP-HPLC following storage at 30° C. for 9 and 14 weeks (see Table 2 of Example 2).

FIG. 4: Stability of liraglutide in the presence of phosphate anions (with and without mannitol) assessed by RP-HPLC following storage at 30° C. for 9 and 14 weeks (see Table 2 of Example 2).

FIG. 5: Stability of liraglutide in the presence of sulphate anions (with or without mannitol) assessed by RP-HPLC following storage at 30° C. for 9 and 14 weeks (see Table 2 of Example 2).

### DESCRIPTION OF THE SEQUENCE LISTINGS

SEQ ID NO: 1: GLP-1(7-37)
SEQ ID NO: 2: GLP-1(7-36)NH2
SEQ ID NO: 3: exendin-4/Exenatide
SEQ ID NO: 4: exendin-3
SEQ ID NO: 5: Albiglutide
SEQ ID NO: 6: Dulaglutide
SEQ ID NO: 7: Liraglutide
SEQ ID NO: 8: Lixisenatide
SEQ ID NO: 9: artificial sequence
SEQ ID NO: 10: artificial sequence
SEQ ID NO: 11: artificial sequence
SEQ ID NO: 12: exemplary serum albumin sequence
SEQ ID NO: 13: exemplary FcIgG4 sequence

### DETAILED DESCRIPTION

As used herein a GLP-1 receptor agonist is any insulinotropic peptide which fully or partially activates the human GLP-1 receptor. In one embodiment, the GLP-1 receptor agonist is any peptide that binds to a GLP-1 receptor with an affinity constant (K_{D}) of below 1 μM, for example below 100 nM as measured by methods known in the art (see for example WO98/08871, the contents of which is herein incorporated by reference) and exhibits insulinotropic activity, where insulinotropic activity may be measured in vivo or in vitro assays known to the skilled person.

In one embodiment, the GLP-1 receptor agonist is an insulinotropic analogue or derivative of GLP-1(7-37) or an insulinotropic analogue or derivative of GLP-1(7-36)NH2. In a particular embodiment, the GLP-1 receptor agonist is an insulinotropic analogue or derivative of GLP-1(7-37). GLP-1(7-37) has the sequence set out in SEQ ID NO: 1. GLP-1(7-36)NH2 has the sequence set out in SEQ ID NO: 2.

An exemplary derivative of GLP-1(7-37) or GLP-1(7-36)NH2 bears one or more lipophilic substituents attached to the parent peptide (GLP-1(7-37) or GLP-1(7-36)NH2), optionally via a linker.

Another exemplary derivative is a fused derivative wherein the parent peptide (GLP-1(7-37) or GLP-1(7-36)NH2) is fused to a protein, e.g. comprising serum albumin or an antibody fragment e.g. the Fc portion of an antibody such as an immunoglobulin G (IgG) e.g. IgG4 optionally via a linker.

Another exemplary derivative is one in which a simple amide (CONH2) is formed of the C terminal COOH group.

Analogues of GLP-1(7-37) or GLP-1(7-36)NH2 comprise sequence changes and exemplary analogues include exendin-4, exendin-3, an exendin-4 analogue, an exendin-3 analogue, an exendin-4 derivative or an exendin-3 derivative. Exendin-4 has the sequence set out in SEQ ID NO: 3. Exendin-3 has the sequence set out in SEQ ID NO: 4.

In one embodiment, the GLP-1 receptor agonist comprises or more suitably consists of a sequence sharing 50%, 55%, 60%, 65%, 70%, 75%, 80%, 85%, 90%, 95%, 98% or greater sequence identity with SEQ ID NO: 1 or SEQ ID NO: 2, suitably with SEQ ID. NO: 1. For example, there are up to 5 e.g. at most 1 or 2 amino acid changes as compared to SEQ ID NO: 1 or SEQ ID NO: 2.

For the purposes of comparing two related polypeptide sequences, the “% sequence identity” between a first polypeptide sequence and a second polypeptide sequence may be calculated using NCBI BLAST v2.0, using standard settings for polypeptide sequences (BLASTP).

In one embodiment, the GLP-1 receptor agonist has or comprises the sequence set out below (SEQ ID NO: 9):

H-X¹-X²-G-T-F-T-S-D-X³-S-X⁴-X⁵-X⁶-E-X⁷-X⁸-A-X⁹-X¹⁰-X″-F-I-X¹²-W-L-X¹³-X¹⁴-G-X¹⁵

wherein
X¹ is A, G or S;
X² is E or D;
X³ is V or L;
X⁴ is S or K;
X⁵ is Y or Q;
X⁶ is L or M;
X⁷ is G or E;
X⁸ is Q or E;
X⁹ is A or V;
X¹⁰ is K or R;
X¹¹ is E or L;
X¹² is A or E;
X¹³ is V or K;
X¹⁴ s K, R or N; and
X¹⁵ is R or G;
   or is a derivative thereof such as a derivative in which a simple amide (CONH2) is formed of the C terminal COOH group and/or a side chain bears a lipophilic substituent, optionally via a linker.

In a further embodiment, the GLP-1 receptor agonist has or comprises the sequence set out below:

[H-X¹-X²-G-T-F-T-S-D-X³-S-X⁴-X⁵-X⁶-E-X⁷-X⁸-A-X⁹-X¹⁰-X″-F-I-X¹²-W-L-X¹³-X¹⁴-G-X¹⁵]ₙ-X¹⁶

wherein X¹-X¹⁵ are as described above for SEQ ID NO: 9;
n is 1 or 2; and
X¹⁶ is absent, G, PSSGAPPPS (SEQ ID NO: 10), PSSGAPPSKKKKKK (SEQ ID NO: 11), a serum albumin sequence or an FcIgG4 sequence;
or is a derivative thereof such as a derivative in which a simple amide (CONH2) is formed of the C terminal COOH group and/or a side chain bears a lipophilic substituent, optionally via a linker.

An exemplary serum albumin sequence is: DAHKSEVAHRFKDLGEENFKALVLIA FAQYLQQCPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFLKK YLYEIARRHPYFYAPELLFFAKRYKAAFTECCQAADKAACLLPKLDELRDEGKASSA KQRLKCASLQKFGERAFKAWAVARLSQRFPKAEFAEVSKLVTDLTKVHTECCHGDL LECADDRADLAKYICENQDSISSKLKECCEKPLLEKSHCIAEVENDEMPADLPSLAAD FVESKDVCKNYAEAKDVFLGMFLYEYARRHPDYSVVLLLRLAKTYETTLEKCCAA ADPHECYAKVFDEFKPLVEEPQNLIKQNCELFEQLGEYKFQNALLVRYTKKVPQVST PTLVEVSRNLGKVGSKCCKHPEAKRMPCAEDYLSVVLNQLCVLHEKTPVSDRVTKC CTESLVNRRPCFSALEVDETYVPKEFNAETFTFHADICTLSEKERQIKKQTALVELVK HKPKATKEQLKAVMDDFAAFVEKCCKADDKETCFAEEGKKLVAASQAALGL (SEQ ID NO: 12) which may optionally be conjugated to the rest of the GLP-1 receptor agonist sequence via a linker sequence of e.g. up to 10 amino acids and may optionally bear a C terminal extension of e.g. up to 10 amino acids.

An exemplary FcIgG4 sequence is: GGGGGSGGGGSGGGGSAESKYGPPCPPC PAPEAAGGPSVFLFPPKPKDTLMISRTPEVTCVVVDVSQEDPEVQFNWYVDGVEVH NAKTKPREEQFNSTYRVVSVLTVLHQDWLNGKEYKCKVSNKGLPSSIEKTISKAKG QPREPQVYTLPPSQEEMTKNQVSLTCLVKGFYPSDIAVEWESNGQPENNYKTTPPVL DSDGSFFLYSRLTVDKSRWQEGNVFSCSVMHEALHNHYTQKSLSLSLG (SEQ ID NO: 13) which may optionally be conjugated to the rest of the GLP-1 receptor agonist sequence via a linker sequence of e.g. up to 10 amino acids and may optionally bear a C terminal extension of e.g. up to 10 amino acids.

Lipophilic substituents as mentioned above include acyl groups such as a straight chain or branched fatty acid. A suitable acyl group typically has the formula CH₃(CH₂)ₙCO—, wherein n=4-38, for example 12-38, such as palmitoyl (i.e. hexadecanoyl). The lipophilic substituent may be attached to the parent peptide (i.e. main GLP-1 receptor agonist sequence) via a linker. A suitable linker is an amino acid, wherein the lipophilic substituent may be attached to the amino acid via the side chain of the amino acid, via the N-terminus of the amino acid, or via the C-terminus of the amino acid. Any amino acid is potentially capable of acting as a linker via connections to both its C-terminus and to its N-terminus. Amino acids may also act as a linker via their side chain moiety. Such amino acids have suitable side chain functionality for forming the linkage e.g. functionality which can react with an acyl groups to form a stable bond. Suitable amino acids include, but are not limited to, lysine, aspartic acid, glutamic acid, serine, threonine, cysteine and tyrosine. In one embodiment the linker is a glutamic acid residue and is suitably linked at one end via the carboxylic acid of its side chain functionality (i.e. the include, but are not limit at the other end by its N-terminus or C-terminus, suitably via its N-terminus. The linker may also be a short peptide sequence e.g. of 2-5 amino acids. In a particular embodiment, the lipophilic substituent (with linker) is a γ-E-palmitoyl group (i.e. a γ-E-hexadecanoyl group).

In embodiments wherein the parent peptide is or comprises (GLP-1(7-37) or GLP-1(7-36)NH2) (SEQ ID NO: 1 or SEQ ID NO: 2, respectively), suitably the lipophilic substituent is attached to the parent peptide via the lysine residue at position 26, optionally via a linker.

In embodiments wherein the parent peptide comprises the sequence set out in SEQ ID NO: 9, the lipophilic substituent is suitably connected via residue X¹⁰, which is suitably a lysine residue, wherein the attachment is optionally via a linker.

In one embodiment, the GLP-1 receptor agonist comprises one or more asparagine or glutamine residues, particularly one or more glutamine residues, and in particular said residue(s) is(are) within the sequence of SEQ ID NO: 9.

In one embodiment, the GLP-1 receptor agonist is selected from the group consisting of albiglutide, dulaglutide, exenatide, liraglutide and lixisenatide. Suitably, the GLP-1 receptor agonist is liraglutide or lixisenatide, in particular liraglutide.

Albiglutide is a GLP-1 dimer fused to human albumin, having the sequence as set out in SEQ ID NO: 5. Marketed under the tradenames Eperzan/Tanzeum, it has a circulating half-life of 4-7 days.

Dulaglutide is a GLP-1 analogue consisting of GLP-1(7-37) covalently linked to an Fc fragment of human IgG4, having the sequence as set out in SEQ ID NO: 6. Marketed under the tradename Trulicity, it has a circulating half-life of approximately 5 days.

Exenatide is a synthetic version of exendin-4, a GLP-1 analogue with 53% sequence identity with GLP-1(7-36)NH2, having the sequence as set out in SEQ ID NO: 3. Marketed under the tradenames Byetta/Bydureon, it has a circulating half-life of 2.4 hours.

Liraglutide ((Arg(34), Lys(26)(N-(γ-Glu(N-palmitoyl)))-GLP-1(7-37)) is an acylated GLP-1 analogue with 97% sequence identity with GLP-1(7-37), having the sequence as set out in SEQ ID NO: 7. Marketed under the tradenames Victoza/Saxenda, it has a circulating half-life of 13 hours.

Lixisenatide is an analogue of exendin-4, where proline is omitted at position 38 and the C-terminal has been modified to add six lysine residues, having the sequence as set out in SEQ ID NO: 8. Marketed under the tradenames Lyxumia/Adlyxin, it has a circulating half-life of 2-4 hours.

The concentration of GLP-1 receptor agonist in the composition is typically between 10 μg/mL and 50 mg/mL, for example between 200 μg/mL and 10 mg/mL, or between 1 mg/mL and 10 mg/mL.

When the GLP-1 receptor agonist is albiglutide, suitably the concentration of albiglutide in the composition is between 5 mg/mL and 200 mg/mL, for example between 50 mg/mL and 150 mg/mL, such as around 100 mg/mL.

When the GLP-1 receptor agonist is dulaglutide, suitably the concentration of dulaglutide in the composition is between 0.5 mg/mL and 20 mg/mL, for example between 1 mg/mL and 5 mg/mL, such as around 1.5 mg/mL or around 3 mg/mL.

When the GLP-1 receptor agonist is exenatide, suitably the concentration of exenatide in the composition is between 0.1 mg/mL and 0.5 mg/mL, for example between 100 μg/mL and 500 μg/mL, such as around 250 μg/mL.

When the GLP-1 receptor agonist is liraglutide, suitably the concentration of liraglutide in the composition is between 0.5 mg/mL and 20 mg/mL, for example between 1 mg/mL and 10 mg/mL, such as around 6 mg/mL.

When the GLP-1 receptor agonist is lixisenatide, suitably the concentration of lixisenatide in the composition is between 0.1 μg/mL and 250 μg/mL, for example between 10 μg/mL and 50 μg/mL, such as around 17 μg/mL or around 33 μg/mL.

The composition of the invention contains multivalent anions as a stabilising agent, wherein the multivalent anions have a charge of at least minus 2 (which may also be written as “−2” or “minus two”). The multivalent anions have a charge of at least minus 2, such as minus 2 (“divalent anions”), minus 3 (“trivalent anions”) or minus 4 (tetravalent anions”). In one embodiment, the multivalent anions have a charge of minus 2 and are divalent anions. In another embodiment, the multivalent anions have a charge of minus 3 and are trivalent anions. In a further embodiment, the multivalent anions are a mixture of anions having charge of minus 2 and anions having charge of minus 3, i.e. are a mixture of divalent and trivalent anions. In another embodiment, the multivalent anions are divalent anions, trivalent anions or a mixture thereof.

The multivalent anions are species which do not comprise any group capable of forming a positive charge (e.g. by protonation) in the range of pH 4-9 at 25° C. Thus, the multivalent anions do not contain basic nitrogen centres, i.e. nitrogen centres which are capable of being protonated. In particular, the multivalent anions do not contain a quaternary ammonium group (i.e. a positively charged tetra-substituted nitrogen atom). The multivalent anions do not contain protonatable nitrogen centres with pKₐ between 5-10 or 3-11 at 25° C. The multivalent anions are not amino acids and particularly are not one of the 20 natural amino acids in L or D form or any mixture thereof (including a racemic mixture). Thus, the multivalent anions are not glutamate, aspartate or a mixture thereof. The multivalent anions are not peptides or proteins (i.e. molecules which comprise two or more amino acid residues).

In one embodiment, the multivalent anions do not contain nitrogen atoms.

The multivalent anions are not nitrogen-containing chelating agents, and in particular are not ethylenediaminetetraacetic acid (EDTA).

Suitably the multivalent anions have a molecular weight of less than 500 Da, for example less than 400 Da, less than 300 Da or less than 200 Da.

If a molecular entity has more than one ionisable group, then it can exist in more than one charged form (i.e. can exist as distinct species with differing charge). In the context of the present invention each form with a different charge is considered to be a separate species which may or may not be a suitable multivalent anion within the context of the invention. For example, citric acid can exist in the following charged states:

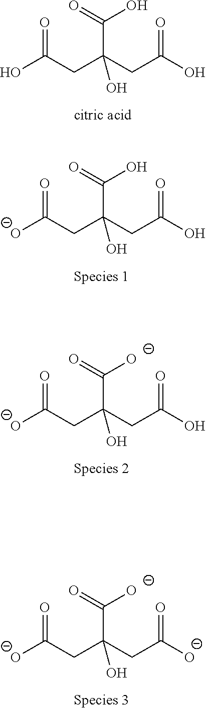 [SMILES: O=C(O)CC(O)(CC(=O)O)C(=O)O ; O=C([O-])CC(O)(CC(=O)O)C(=O)O ; O=C([O-])CC(O)(CC(=O)O)C(=O)[O-] ; O=C([O-])CC(O)(CC(=O)[O-])C(=O)[O-]]

Only divalent citric-acid based species (“Species 2” in the figure above) with a charge of minus 2 and trivalent citric-acid based species (“Species 3” in the figure above) with a charge of minus 3 are multivalent anions according to the present invention. Conversely, citric acid with a charge of 0, and monovalent citric acid-based species (“Species 1” in the figure above) with a charge of minus 1 are not multivalent anions according to the present invention. In one embodiment, the multivalent anions are divalent citrate anions. In another embodiment, the multivalent anions are trivalent citrate anions. In a further embodiment, the multivalent anions are selected from divalent citrate anions, trivalent citrate anions and mixtures thereof. In a further embodiment, the multivalent anions are a mixture of divalent and trivalent citrate anions.

Monovalent sulphate anions (also known as a bisulfate anions (HSO₄⁻)) have a charge of minus 1 therefore are not multivalent anions according to the present invention. Divalent sulphate anions (SO₄²⁻) have a charge of minus 2 and are multivalent anions according to the present invention. In one embodiment the multivalent anions are divalent sulphate anions.

Phosphoric acid ionizes to give the following species:

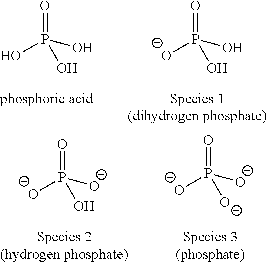 [SMILES: O=P(O)(O)O ; O=P([O-])(O)O ; O=P([O-])([O-])O ; O=P([O-])([O-])[O-]]

Only divalent phosphoric acid-based species (“Species 2” in the figure above) with a charge of minus 2 and trivalent phosphoric acid-based species (“Species 3” in the figure above) with a charge of minus 3 are multivalent anions according to the present invention. Conversely, phosphoric acid with a charge of 0, and monovalent phosphoric acid-based species (“Species 1” in the figure above) with a charge of minus 1 are not multivalent anions according to the present invention. In one embodiment, the multivalent anions are divalent phosphate anions. In another embodiment, the multivalent anions are trivalent phosphate anions. In a further embodiment, the multivalent anions are selected from divalent phosphate anions, trivalent phosphate anions and mixtures thereof. In a further embodiment, the multivalent anions are a mixture of divalent and trivalent phosphate anions.

It should be noted that where multivalent anions with charge of at least minus 2 are present, this does not preclude other related species which are not multivalent anions according to the invention from being present in the composition. For example, in the case of the composition of the invention containing divalent and/or trivalent phosphate anions (HPO₄²⁻ anions and/or PO₄³⁻ anions), phosphoric acid and/or monovalent phosphate anions (H₂PO₄⁻) may also be present in the composition, but do not contribute to the “multivalent anions” requirement of the invention.

Multivalent anions are conjugate bases of a conjugate acid-base pair. They can be formed from the conjugate acid by addition of a strong base, resulting in a loss of a proton. Therefore, in one embodiment the multivalent anions with charge of at least minus 2 are added to the composition in the form of the conjugate acid and pH is adjusted to a level which results in a loss of a proton and formation of the conjugate base. For example, citric acid is added to the composition, followed by adjustment of pH to a level where citrate multivalent anions are formed. More commonly, the multivalent anions originate from a salt dissociation. Thus, in one embodiment the multivalent anions with charge of at least minus 2 are added to the composition in salt form, for example as a sodium salt, a potassium salt, a calcium salt or a magnesium salt.

In one embodiment the multivalent anions are selected from divalent citrate anions, trivalent citrate anions, divalent sulphate anions, divalent phosphate anions, trivalent phosphate anions and mixtures thereof. In another embodiment, the multivalent anions are selected from divalent citrate anions, trivalent citrate anions, divalent sulphate anions, and mixtures thereof. In another embodiment, the multivalent anions are selected from divalent citrate anions, trivalent citrate anions, divalent phosphate anions, trivalent phosphate anions and mixtures thereof. In another embodiment, the multivalent anions are selected from divalent phosphate anions, trivalent phosphate anions, divalent sulphate anions and mixtures thereof. Thus, reference to “multivalent anions” includes mixtures. In one embodiment, the multivalent anions are a mixture of at least two different multivalent anions having a charge of at least minus 2.

The multivalent anions are present in the composition at a total concentration of at least 15 mM, for example at least 20 mM, at least 30 mM, at least 40 mM or at least 50 mM. In one embodiment, the multivalent anions are present at a total concentration of 15-150 mM, such as 15-120 mM, 20-120 mM, 20-100 mM, 20-80 mM or around 50 mM. For the avoidance of doubt, where the multivalent anions are a mixture of anions, the concentration is the total sum concentration of every multivalent anion having a charge of at least minus 2 present in the composition. Hence, the total concentration of multivalent anions may also be described as the “combined concentration” of multivalent anions.

The concentration of multivalent anions in the composition is calculated based on the concentration of the substance contributing the multivalent anions, the pH of the solution and the pKₐ values of the ionisable groups on each species present in the composition.

By way of illustration, phosphoric acid is a triprotic acid and dissociates in solution in three steps as shown in the following formulae:

H₃PO₄+H₂O=H₂PO₄⁻+H₃O⁺

Kₐ₁=[H₂PO₄⁻][H₃O⁺]/[H₃PO₄]

H₂PO₄^{-+H}₂O=HPO₄²⁻+H₃O⁺

Kₐ₂=[HPO₄²⁻][H₃O⁺]/[H₂PO₄^{-]}

HPO₄²⁻+H₂O=PO₄³⁻+H₃O⁺

Kₐ₃=[PO₄³⁻][H₃O⁺]/[HPO₄²⁻]

The sum of the concentrations of the four phosphorus containing species after equilibration at the appropriate pH is the same as the concentration of phosphorus containing species added (this is a requirement for mass balance of phosphorus). [H₃O⁺] can be derived from the pH and the Kₐ₁, Kₐ₂ and Kₐ₂ values can be derived from the known values for pKₐ₁, pKₐ₂ and pKₐ₃.

Thus, provided that the pH of the composition, the concentration of starting materials and the pKₐ values of ionisable groups are known, a skilled person can calculate the concentrations of each of the anions including the multivalent anions.

For the avoidance of doubt, the multivalent anions cannot be the same species as the GLP-1 receptor agonist.

In one embodiment, the aqueous solution composition of the invention does not contain species which comprise groups capable of forming a positive charge in the range of pH 4-9. In one embodiment, the aqueous solution composition of the invention does not contain species comprising basic nitrogen centres i.e. nitrogen centres which are capable of being protonated. For the avoidance of doubt, the GLP-1 receptor agonist is not considered as a “species” in this context. In one embodiment, the aqueous solution composition of the invention does not contain species comprising protonatable nitrogen centres with pKₐ between 5-10 or 3-11 at 25° C. Again for the avoidance of doubt, the GLP-1 receptor agonist is not considered as a “species” in this context. In one embodiment, the aqueous solution composition does not contain an amino acid. In another embodiment, the aqueous solution composition does not contain a positively charged additive. In one embodiment, the aqueous solution composition does not contain an additive which bears a positive charge in the range of pH 4-9 at 25° C. In one embodiment, the aqueous solution composition does not contain propylene glycol. In one embodiment, the aqueous solution composition does not contain nitrogen-containing chelating agents, in particular EDTA.

The pH of the composition should be suitable for the particular GLP-1 receptor agonist. For example, for a composition containing exenatide, the pH of the composition is suitably between 4 and 5, for example about 4.5. For a composition containing liraglutide, the pH of the composition is suitably between 7.5 and 8.5, for example about 8.1. In one embodiment, the pH of the composition is between 4 and 9.

The compositions of the invention may additionally comprise a preservative such as a phenolic or a benzylic preservative. The preservative is suitably selected from the group consisting of phenol, m-cresol, chlorocresol, chlorophenol, benzyl alcohol, propyl paraben, methyl paraben, benzalkonium chloride and benzethonium chloride, in particular phenol, m-cresol and benzyl alcohol.

The concentration of preservative is typically 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

The composition may additionally comprise a surfactant. In one embodiment, the surfactant is a non-ionic surfactant. In another embodiment, the surfactant is a cationic surfactant.

A particularly suitable class of non-ionic surfactants is the polysorbates (fatty acid esters of ethoxylated sorbitan), such as polysorbate 20. Polysorbate 20 is a mono ester formed from oleic acid and polyoxyethylene (20) sorbitan in which the number 20 indicates the number of oxyethylene groups in the molecule. Polysorbate 20 is known under a range of brand names including in particular Tween 20, and also Alkest TW 20. Other suitable polysorbates include polysorbate 40, polysorbate 60 and polysorbate 80.

Another suitable class of non-ionic surfactants is the alkyl glycosides, especially dodecyl maltoside. Other alkyl glycosides include dodecyl glucoside, octyl glucoside, octyl maltoside, decyl glucoside, decyl maltoside, tridecyl glucoside, tridecyl maltoside, tetradecyl glucoside, tetradecyl maltoside, hexadecyl glucoside, hexadecyl maltoside, sucrose monooctanoate, sucrose mono decanoate, sucrose monododecanoate, sucrose monotridecanoate, sucrose monotetradecanoate and sucrose monohexadecanoate.

Another suitable class of non-ionic surfactants is block copolymers of polyethylene glycol and polypropylene glycol, also known as poloxamers, especially poloxamer 188, poloxamer 407, poloxamer 171 and poloxamer 185. Poloxamers are also known under brand names Pluronics or Koliphors. For example, poloxamer 188 is marketed as Pluronic F-68.

Another suitable class of non-ionic surfactants is alkyl ethers of polyethylene glycol, especially those known under a brand name Brij, such as selected from polyethylene glycol (2) hexadecyl ether (Brij 52), polyethylene glycol (2) oleyl ether (Brij 93) and polyethylene glycol (2) dodecyl ether (Brij L4). Other suitable Brij surfactants include polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

Another suitable class of non-ionic surfactants are alkylphenyl ethers of polyethylene glycol, especially 4-(1,1,3,3-tetramethylbutyl)phenyl-polyethylene glycol, also known under a brand name Triton X-100.

Suitable cationic surfactants include benzalkonium and benzethonium salts. In one embodiment, the cationic surfactant is selected from benzethonium salts e.g. benzethonium halide such as benzethonium chloride. In another embodiment, the cationic surfactant is selected from benzalkonium salts e.g. benzalkonium halide such as benzalkonium chloride. In a further embodiment, the cationic surfactant is a mixture of benzethonium salts and benzalkonium salts such as a mixture of benzethonium chloride and benzalkonium chloride.

When included, the concentration of the surfactant in the formulation will typically be in the range of 1-2000 μg/ml, e.g. 5-1000 μg/ml, e.g. 10-500 μg/ml, such as 10-200 μg/ml.

The compositions of the invention can have a wide range of osmolarity, and may be hypotonic, isotonic or hypertonic. Suitably, the composition of the invention is substantially isotonic. Suitably the osmolarity of the composition is selected to minimize pain according to the route of administration e.g. upon injection. Suitably the composition has an osmolarity of 50-1000 mOsm/L, such as 100-500 mOsm/L, for example about 300 mOsm/L.

The composition may additionally comprise a tonicity modifier, which may be charged or uncharged. Examples of uncharged tonicity modifiers include glycerol, mannitol, propylene glycol, trehalose, PEG300 and PEG400. When included, an uncharged tonicity modifier is typically added at a concentration of 50-1000 mM, for example 100-500 mM, such as about 300 mM. Examples of charged tonicity modifiers include sodium chloride, sodium sulphate, sodium acetate, sodium lactate, and amino acids such as glycine or arginine. When included, a charged tonicity modifier is typically added at a concentration of 25-500 mM, for example 50-250 mM such as about 150 mM. In certain cases, if the concentration of multivalent anions with a charge of at least minus 2 is sufficiently high then they can also serve as a tonicity modifier.

The composition may comprise a buffer. Suitable buffers include acetate, succinate, maleate, phosphate and TRIS. When included, the buffer is typically present at a concentration of 0.5-50 mM, such as 1-20 mM, for example 2-5 mM. In certain cases, the multivalent anions having a charge of at least minus 2 can also serve as a buffer.

The composition may comprise additional active ingredients. In one embodiment, the composition of the invention additionally comprises glucagon or a long acting insulin. Examples of long acting insulins include insulin glargine and insulin degludec. In one embodiment, the composition of the invention additionally comprises peptide YY (peptide tyrosine tyrosine) or an analogue thereof. In one embodiment, the GLP-1 receptor agonist is the only active ingredient in the composition.

The following specific embodiments of the invention are envisaged. In one embodiment is provided an aqueous solution composition comprising a GLP-1 receptor agonist which has or comprises the sequence set out in SEQ ID NO: 9 (wherein X¹-X¹⁵ are as defined above) as an active ingredient, and multivalent anions selected from divalent citrate anions, trivalent citrate anions, divalent sulphate anions, divalent phosphate anions, trivalent phosphate anions and mixtures thereof, as stabilising agent, wherein the total concentration of multivalent anions in the composition is at least 15 mM, at least 20 mM, at least 30 mM, at least 40 mM or at least 50 mM. In another embodiment is provided an aqueous solution composition comprising a GLP-1 receptor agonist selected from albiglutide, dulaglutide, exenatide, liraglutide and lixisenatide as an active ingredient, and multivalent anions selected from divalent citrate anions, trivalent citrate anions, divalent sulphate anions, divalent phosphate anions, trivalent phosphate anions and mixtures thereof, as stabilising agent, wherein the total concentration of multivalent anions in the composition is at least 15 mM, at least 20 mM, at least 30 mM, at least 40 mM or at least 50 mM. In another embodiment is provided an aqueous solution composition comprising a GLP-1 receptor agonist which is liraglutide as an active ingredient, and multivalent anions selected from divalent citrate anions, trivalent citrate anions and mixtures thereof as stabilising agent wherein the total concentration of multivalent anions in the composition is at least 15 mM, at least 20 mM, at least 30 mM, at least 40 mM or at least 50 mM. In another embodiment is provided an aqueous solution composition comprising a GLP-1 receptor agonist which is liraglutide as an active ingredient, and multivalent anions which are divalent sulphate anions as stabilising agent wherein the total concentration of multivalent anions in the composition is at least 15 mM, at least 20 mM, at least 30 mM, at least 40 mM or at least 50 mM. In another embodiment is provided an aqueous solution composition comprising a GLP-1 receptor agonist which is liraglutide as an active ingredient, and multivalent anions which are divalent sulphate anions as stabilising agent, at a concentration of at least 15 mM, at least 20 mM, at least 30 mM, at least 40 mM or at least 50 mM. In another embodiment is provided an aqueous solution composition comprising a GLP-1 receptor agonist which is liraglutide as an active ingredient, and multivalent anions selected from divalent phosphate anions, trivalent phosphate anions and mixtures thereof as stabilising agent wherein the total concentration of multivalent anions in the composition is at least 15 mM, at least 20 mM, at least 30 mM, at least 40 mM or at least 50 mM.

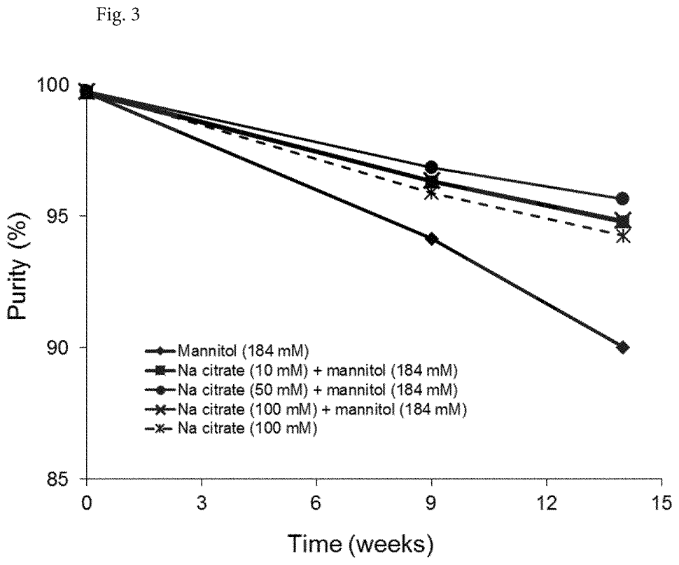

The presently claimed invention derives from the surprising observation that compositions containing a GLP-1 receptor agonist as active ingredient, in particular liraglutide, are stabilized by the addition of multivalent anions, particularly divalent and trivalent anions. Having recognized the need to improve the stability of currently marketed formulations containing GLP-1 receptor agonists, the present inventors began their investigations by using a composition based on Victoza (liraglutide), wherein the tonicity modifier propylene glycol was replaced with various alternative tonicity modifiers. Stability (primarily chemical stability) was determined with respect to product purity assessed by reversed-phase HPLC. As explained in Example 1, replacement of the propylene glycol with mannitol, trehalose or sodium chloride was observed to have minimal impact on stability of the liraglutide. The addition of histidine was observed to impair the stability of liraglutide. The tonicity modifier propylene glycol was then replaced with various multivalent anions. As can be seen from Example 2 and FIGS. 3-5, the presence of multivalent anions with a charge of at least minus 2 led to a considerable improvement in the stability of liraglutide following storage at 30° C. for 9 weeks and 14 weeks.

GLP-1 receptor agonists share very similar instability pathways. Whilst the physical instability of these products comprises formation of soluble aggregates and insoluble aggregates (non-specifically structured particles and structured amyloid fibrils), the sites of chemical instability are limited to asparagine (N) or glutamine (Q) residues that are prone to the formation of cyclic imide structures, which are in turn cleaved to asparte or isoaspartate residues (in the case of asparagine) or glutamate or isoglutamate residues (in the case of glutamine). These processes are referred to as aspartate/glutamate deamidation. Although the cyclic imide structure is an intermediate in this process, it is often a relatively stable impurity for this type of therapeutic peptide. The chemically related impurities of GLP-1 receptor agonists are thus typically represented by cyclic imides, and molecules where glutamine/asparagine have turned into the respective acids or iso-acids. Without being bound by theory, it is believed that the surprising stabilising effect of multivalent anions is due at least in part to their binding to the structure of the GLP-1 receptor agonist, resulting in formation of a tighter peptide structure which in turn limits the exposure of the deamidation sights to the solvent, thus limiting the rate of deamidation. Given the high degree of homology shared amongst the GLP-1 receptor agonists, very similar interactions are expected between their respective structures and the multivalent anions. Consequently, it is considered likely that the improvement in stability observed for liraglutide upon addition of multivalent anions with charge of at least minus 2 will also be observed for GLP-1 receptor agonists generally, in particular exenatide, dulaglutide, lixisenatide and albiglutide.

Chemical stability of a GLP-1 receptor composition can be evaluated by reversed phase HPLC (RP-HPLC) to determine the proportion of chemically related GLP-1 receptor agonist species (i.e. species generated during storage or other stress conditions by chemical modification of GLP-1 receptor agonist, including deamidation or cyclic imide formation) as described in the General Procedures. This type of RP-HPLC experiment can be used to determine the purity of the GLP-1 receptor agonist species i.e. the amount of unaltered GLP-1 receptor agonist (e.g. as a % by total weight of the original GLP-1 receptor agonist) following storage or other stress conditions.

Suitably, a composition of the invention retains at least 90%, e.g. at least 91%, at least 92%, at least 93%, at least 94%, at least 95%, at least 96%, at least 97%, at least 98% or at least 99% purity (i.e. native GLP-1 receptor agonist (by total weight of GLP-1 receptor agonist in the composition at time T=0)) following storage at 15-30° C. e.g. 30° C., for at least 3 weeks, for example for 3 weeks, 4 weeks, 5 weeks, 6 weeks, 7 weeks, 8 weeks, 9 weeks, 10 weeks, 11 weeks, 12 weeks, 13 weeks or 14 weeks.

Suitably, a composition of the invention retains at least 95%, e.g. at least 96%, at least 97%, at least 98% or at least 99% purity (i.e. native GLP-1 receptor agonist (by total weight of GLP-1 receptor agonist in the composition at time T=0)) following storage for 9 weeks at 30° C.

Suitably, a composition of the invention retains at least 90%, e.g. at least 91%, at least 92%, at least 93%, at least 94%, at least 95%, at least 96%, at least 97%, at least 98% or at least 99% purity (i.e. native GLP-1 receptor agonist (by total weight of GLP-1 receptor agonist in the composition at time T=0)) following storage for 14 weeks at 30° C.

Suitably, a composition of the invention retains at least 90%, e.g. at least 91%, at least 92%, at least 93%, at least 94%, at least 95%, at least 96%, at least 97%, at least 98% or at least 99% purity (i.e. native GLP-1 receptor agonist (by total weight of GLP-1 receptor agonist in the composition at time T=0)) following storage at 32° C. or higher, for example at 32° C., 34° C., 35° C., 36° C., 37° C., 38° C. or 40° C., for at least 3 weeks, for example for 3 weeks, 4 weeks, 5 weeks, 6 weeks, 7 weeks, 8 weeks, 10 weeks, 12 weeks or 14 weeks.

Suitably, a composition of the invention retains at least 90%, e.g. at least 91%, at least 92%, at least 93%, at least 94%, at least 95%, at least 96%, at least 97%, at least 98% or at least 99% purity (i.e. native GLP-1 receptor agonist (by total weight of GLP-1 receptor agonist in the composition at time T=0)) following storage at 25° C. for at least 3 months, such as 3 months, 6 months, 12 months or 24 months; and also retains at least 90%, e.g. at least 91%, at least 92%, at least 93%, at least 94%, at least 95%, at least 96%, at least 97%, at least 98% or at least 99% purity (i.e. native GLP-1 receptor agonist (by total weight of GLP-1 receptor agonist in the composition at time T=0)) following an in-use period of 28 days at 30° C., starting immediately after the end of the storage period.

Suitably, a composition of the present invention should exhibit an increase in chemically related GLP-1 receptor agonist species during storage which is at least 10% lower, preferably at least 25% lower, more preferably at least 50% lower, than a composition lacking multivalent anions having charge of at least minus 2 as a stabilising agent, but otherwise identical, following storage under the same conditions and length of time.

Suitably, the proportion of total chemically related GLP-1 receptor agonist species remains below 10% (by weight), e.g. below 9%, 8%, 7%, 6%, 5%, 4%, 3%, 2%, or below 1% during storage at 15-30° C. e.g. 30° C., for at least 4 weeks, for example 4 weeks, 5 weeks, 6 weeks, 7 weeks, 8 weeks, 9 weeks, 10 weeks, 11 weeks, 12 weeks, 13 weeks or 14 weeks.

Suitably, the proportion of total chemically related GLP-1 receptor agonist species remains below 5% (by weight), e.g. below 4%, 3%, 2%, or below 1% during storage at 30° C. for 9 weeks.

Suitably, the proportion of total chemically related GLP-1 receptor agonist species remains below 10% (by weight), e.g. below 9%, 8%, 7%, 6%, 5%, 4%, 3%, 2%, or below 1% during storage at 30° C. for 14 weeks.

Suitably, the proportion of total chemically related GLP-1 receptor agonist species remains below 5% (by weight), e.g. below 4%, 3%, 2%, or 1%, during storage for at least 3 weeks such as at least 4 weeks, at temperatures greater than 30° C., for example during storage for 3 weeks, 4 weeks, 5 weeks, 6 weeks, 7 weeks, 8 weeks or 9 weeks at 32° C., 34° C., 35° C., 37° C., 38° C. or 40° C.

Suitably, the proportion of total chemically related GLP-1 receptor agonist species remains below 10% (by weight), e.g. below 9%, 8%, 7%, 6%, 5%, 4%, 3%, 2%, or 1% during storage at least 32° C. or higher, for example at 32° C., 34° C., 35° C., 37° C., 38° C. or 40° C., for at least 4 weeks, for example for 4 weeks, 5 weeks, 6 weeks, 7 weeks, 8 weeks, 9 weeks, 10 weeks, 11 weeks, 12 weeks, 13 weeks or 14 weeks.

In another aspect of the invention, there is provided the use of multivalent anions having a charge of at least minus 2 and suitably wherein the total concentration of multivalent anions in the composition having a charge of at least minus 2 is at least 15 mM, for stabilizing an aqueous solution composition of a GLP-1 receptor agonist. In particular, the chemical stability of the GLP-1 receptor agonist is improved.

In a further aspect of the invention, there is provided a method of improving the stability (i.e. chemical and/or physical stability) of an aqueous solution composition comprising a GLP-1 receptor agonist as an active ingredient which comprises adding multivalent anions having a charge of at least minus 2 to the composition and suitably wherein the total concentration of multivalent anions in the composition having a charge of at least minus 2 is at least 15 mM. In particular, the chemical stability of the GLP-1 receptor agonist is improved.

In a further aspect of the invention, there is provided a method for increasing the shelf-life of an aqueous solution composition comprising a GLP-1 receptor agonist as an active ingredient which comprises adding multivalent anions having a charge of at least minus 2 to the composition and suitably wherein the total concentration of multivalent anions in the composition having a charge of at least minus 2 is at least 15 mM.

The physical stability of a GLP-1 receptor agonist composition refers to the tendency of the agonist molecule to form soluble aggregates or insoluble aggregates (particles or fibrils) due to, inter alia, destabilizing interactions with surfaces and interfaces, and temperature fluctuations. Physical stability of the GLP-1 receptor agonist composition can be evaluated by methods known in the art, including by visual inspection, size exclusion chromatography (SEC), electrophoresis, analytical ultracentrifugation, light scattering, dynamic light scattering, static light scattering or field flow fractionation. Fibril formation can be assessed by Thioflavin T (ThT) assay. In the present Examples, all aqueous solution compositions of the invention were observed to have good physical stability as no aggregates (soluble or insoluble) were observed.

The chemical stability of a GLP-1 receptor composition refers in particular to the stability with respect to formation of chemically related GLP-1 receptor agonist species (i.e. species generated during storage or other stress conditions by chemical modification of GLP-1 receptor agonist, including deamidation or cyclic imide formation).

Suitably the composition of the invention remains as a clear solution for a longer period of time compared to the current commercially available GLP-1 receptor agonist products, allowing for a longer in-use period. Thus, in one embodiment, the composition of the invention remains as a clear solution during storage at 15-30° C. e.g. at 30° C. for longer than 4 weeks, for example for 5 weeks, 6 weeks, 7 weeks, 8 weeks, 9 weeks, 10 weeks or 12 weeks. By “clear solution” it is meant that no visible precipitation is observed during storage.

Suitably the composition of the invention remains as a clear solution when exposed to temperatures which are higher than those recommended for the current commercially available GLP-1 receptor agonist products. Thus, in one embodiment, the composition of the invention remains as a clear solution during storage for 4 weeks, at temperatures greater than 30° C., for example during storage for 4 weeks at 32° C., at 34° C., at 35° C., at 37° C., at 38° C. or at 40° C.

In one embodiment, the composition according to the invention is a clear solution with low viscosity (e.g. dynamic viscosity of less than 20 cP, such as less than 10 cP, e.g. less than 5 cP at 25° C. measured using a microfluidics capillary extrusion viscometer, such as m-VROC™, RheoSense Inc.).

Suitably the composition of the invention has improved storage stability at increased temperature, while maintaining the in-use stability. Thus, in one embodiment, the composition of the invention remains as a clear solution during storage at 25° C. for at least 3 months, for example 3 months, 6 months, 12 months or 24 months; and also remains as a clear solution during an in-use period of 14 days or 28 days at 30° C., starting immediately after the end of the storage period.

In one embodiment, a composition of the present invention exhibits an increase in high molecular weight species during storage which is at least 10% lower, preferably at least 25% lower, more preferably at least 50% lower, than a composition lacking multivalent anions having a charge of at least minus 2 as a stabilising agent, but otherwise identical (and particularly lacking multivalent anions in the composition having a charge of at least minus 2 at a concentration of at least 15 mM) following storage under the same conditions and length of time.

The composition of the invention is a therapeutic composition (i.e. a composition for use in therapy), and is particularly suitable for the treatment or prevention of a disease or disorder caused by, associated with and/or accompanied by disturbances in carbohydrate and/or lipid metabolism, e.g. for the treatment or prevention of type 1 diabetes, type 2 diabetes, hyperglycaemia, impaired glucose tolerance, obesity or metabolic syndrome. Thus, in one embodiment is provided a method of treating or preventing a disease or disorder caused by, associated with and/or accompanied by disturbances in carbohydrate and/or lipid metabolism, e.g. for the treatment or prevention of type 1 diabetes, type 2 diabetes, hyperglycaemia, impaired glucose tolerance, obesity or metabolic syndrome, which comprises administering to a subject in need thereof a therapeutically effective amount of a composition as described herein. There is also provided a composition as described herein for use as a pharmaceutical, especially for use in treating or preventing diseases or disorders caused by, associated with and/or accompanied by disturbances in carbohydrate and/or lipid metabolism, e.g. for the treatment or prevention of type 1 diabetes, type 2 diabetes, hyperglycaemia, impaired glucose tolerance, obesity or metabolic syndrome.

The therapeutic compositions of the invention are suitably intended for a daily administration, particularly for treatment of type 2 diabetes. The compositions may also be used for less frequent administration, such as twice-weekly, weekly or monthly administration.

There is also provided a container, for example made of plastics or glass, containing one dose or a plurality of doses of the composition as described herein. The container can be for example, a vial, or a cartridge designed to be a replaceable item for use with an injection device.

The compositions of the invention may suitably be packaged for injection, especially sub-cutaneous or intramuscular injection. Sub-cutaneous injection is preferred. Injection may be by conventional syringe or more preferably via a pen device adapted for use by diabetic subjects. Exemplary pen devices include OptiClick®, SoloSTAR® and KwikPen®.

An aspect of the invention is an injection device, particularly a device adapted for subcutaneous or intramuscular injection, for single or multiple-use comprising a container containing one dose or a plurality of doses of the composition of the invention together with an injection needle. In an embodiment the container is a replaceable cartridge which contains a plurality of doses. In an embodiment, the needle is replaceable e.g. after each occasion of use. In one embodiment, the injection device is in the form of a pen. In one embodiment, the injection device is in the form of a pump. In one embodiment, the injection device comprises a pump.

Another aspect of the invention is a medical device comprising a reservoir comprising a plurality of doses of the composition of the invention and a pump adapted for automatic or remote operation such that upon automatic or remote operation one or more doses of the composition of the invention is administered to the body e.g. subcutaneously or intramuscularly. Such devices may be worn on the outside of the body or implanted in the body.

Further aspects include:

An aqueous solution composition comprising liraglutide as an active ingredient and ions selected from citrate ions, phosphate ions and sulphate ions (e.g. contributed by added sodium citrate, sodium phosphate and/or sodium sulphate) at a concentration of at least 15 mM, 20 mM, 30 mM, 40 mM, 50 mM, or 100 mM wherein the pH of the composition is 8-8.5 e.g. 8.1-8.2. For example, the concentration of ions selected from citrate ions, phosphate ions and sulphate ions does not exceed 150 mM e.g. does not exceed 120 mM.

An aqueous solution composition comprising liraglutide as an active ingredient and citrate ions (e.g. contributed by added sodium citrate) at a concentration of at least 15 mM, 20 mM, 30 mM, 40 mM, 50 mM, or 100 mM wherein the pH of the composition is 8-8.5 e.g. 8.1-8.2. For example, the concentration of citrate ions does not exceed 150 mM e.g. does not exceed 120 mM. The composition may contain phosphate ions (e.g. contributed by added sodium phosphate) at a concentration of e.g. 5-10 mM.

An aqueous solution composition comprising liraglutide as an active ingredient and sulphate ions (e.g. contributed by added sodium sulphate) at a concentration of at least 15 mM, 20 mM, 30 mM, 40 mM, 50 mM, or 100 mM wherein the pH of the composition is 8-8.5 e.g. 8.1-8.2. For example, the concentration of sulphate ions does not exceed 150 mM e.g. does not exceed 120 mM. The composition may contain phosphate ions (e.g. contributed by added sodium phosphate) at a concentration of e.g. 5-10 mM.

An aqueous solution composition comprising liraglutide as an active ingredient and phosphate ions (e.g. contributed by added sodium phosphate) at a concentration of at least 15 mM, 20 mM, 30 mM, 40 mM, 50 mM, or 100 mM wherein the pH of the composition is 8-8.5 e.g. 8.1-8.2. For example, the concentration of phosphate ions does not exceed 150 mM e.g. does not exceed 120 mM.

GLP-1 receptor agonist-containing compositions according to the invention are expected to have one or more of the advantages of:
good stability, in particular chemical stability, during the in-use period at temperatures up to 40° C., e.g. up to 30° C.;
good stability, in particular chemical stability, during an extended in-use period e.g. up to 12 weeks;
good storage stability, in particular chemical storage stability, at an increased temperature e.g. 20-25° C. whilst retaining good in-use stability, in particular chemical in-use stability;

Compositions according to the invention are expected to have good chemical and/or physical stability as described herein, in particular good chemical stability.

### EXAMPLES

General Procedures

Reversed Phase High-Performance Liquid Chromatography (RP-HPLC)

Ultra-high performance reverse phase chromatography was performed using the Waters ACQUITY H-class Bio UPLC® system with a 1.7 μm Ethylene Bridged Hybrid particle, 130 Å pore resin trifunctionally immobilised with a C18 ligand in a 50 mm by 2.1 mm column. Insulin samples were bound in an 82% w/v Na₂SO₄, 18% v/v acetonitrile, pH 2.3 mobile phase and eluted in 50% w/v Na₂SO₄, 50% v/v acetonitrile gradient flow. 2 μl of sample was acidified with 0.01M HCl and analysed at 0.61 mL/min, with 214 nm UV detection. All analyses were performed at 40° C.

### Example 1 (Comparative)

### Effect of Additives on the Stability of Liraglutide

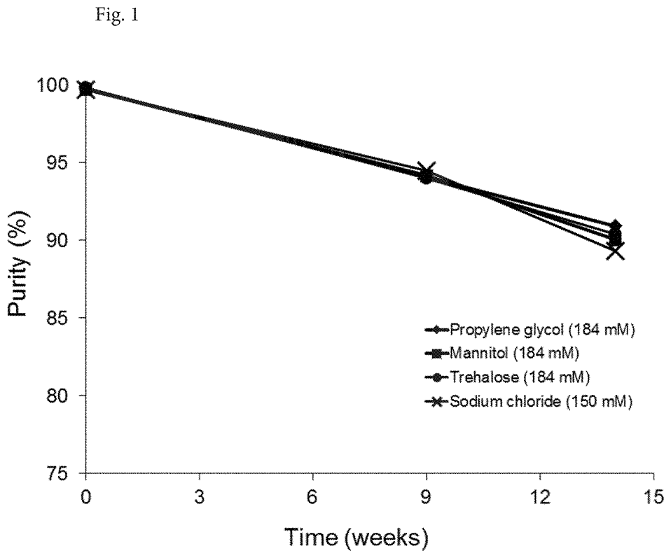

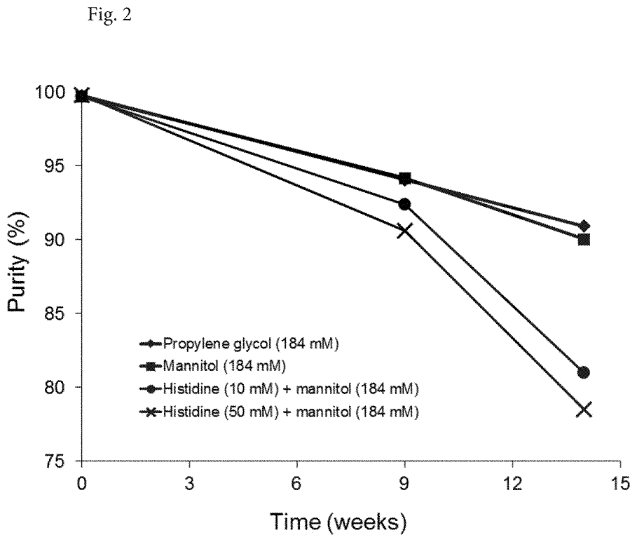

The effect of various tonicity modifiers on the stability of liraglutide was investigated by replacing propylene glycol in the composition of the marketed liraglutide product (Victoza) with two alternative uncharged tonicity modifiers (mannitol and trehalose), one charged tonicity modifier (sodium chloride) and one amino acid (histidine) and assessing purity by RP-HPLC (see General Procedures) following storage at 30° C. The remainder of the composition was identical to that of the Victoza product (liraglutide (6 mg/ml), sodium phosphate (8 mM), phenol (58.4 mM), pH 8.15). It was shown (Table 1 and FIG. 1) that replacing propylene glycol with mannitol, trehalose or sodium chloride had minimal impact on the stability of liraglutide. Histidine, both at 10 mM and at 50 mM concentration, impaired the stability of liraglutide (Table 1 and FIG. 2).

TABLE 1
Purity of liraglutide assessed by visual assessment following storage at
30° C. for 9 and 14 weeks. All formulations were adjusted to pH 8.15
and contained liraglutide (6 mg/ml), sodium phosphate (8 mM),
phenol (58.4 mM) and additive(s) specified in the Table.
Purity was assessed by RP-HPLC.

| | MVA* | Purity (assessed as % RP- | | |
|---|---|---|---|---|
| | conc. | HPLC main peak) | | |
| Additive | (mM) | 0 weeks | 9 weeks | 14 weeks |
| Propylene glycol (184 mM) | 7.3 | 99.75 | 94.05 | 90.91 |
| Mannitol (184 mM) | 7.3 | 99.70 | 94.15 | 90.02 |
| Trehalose (184 mM) | 7.3 | 99.79 | 94.01 | 90.39 |
| Sodium chloride (150 mM) | 7.3 | 99.69 | 94.45 | 89.33 |
| Histidine (10 mM) + Mannitol | 7.3 | 99.69 | 92.39 | 80.98 |
| (184 mM) | | | | |
| Histidine (50 mM) + Mannitol | 7.3 | 99.81 | 90.62 | 78.53 |
| (184 mM) | | | | |

*Multivalent anions having a charge of at least minus 2 (based on pKₐ values of phosphate of 2.2, 7.2 and 12.3 (25° C.); CRC Handbook of Chemistry and Physics, 79^{th} Edition, 1998, D. R. Lide)

### Example 2

### Effect of Multivalent Anions on the Stability of Liraglutide

The effect of multivalent anions on the stability of liraglutide was investigated by adding the various anions to a composition comprising liraglutide (6 mg/ml), sodium phosphate (8 mM), phenol (58.4 mM) at pH 8.15, and assessing purity by RP-HPLC following storage at 30° C. The effect was investigated in the presence of mannitol as a tonicity modifier. In some cases, particularly if the concentration of the multivalent anion was high, the effect was also investigated in the absence of mannitol. Stability of the compositions containing the multivalent anions was compared with that of the compositions containing mannitol only or propylene glycol only (i.e. the composition of the marketed Victoza product).

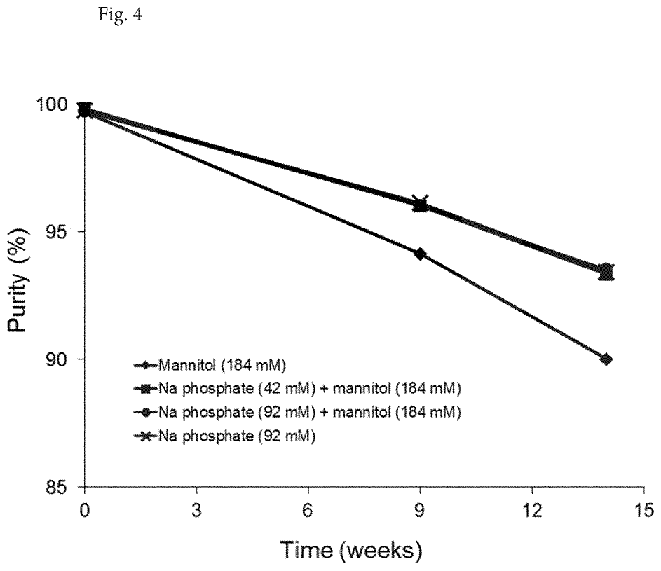

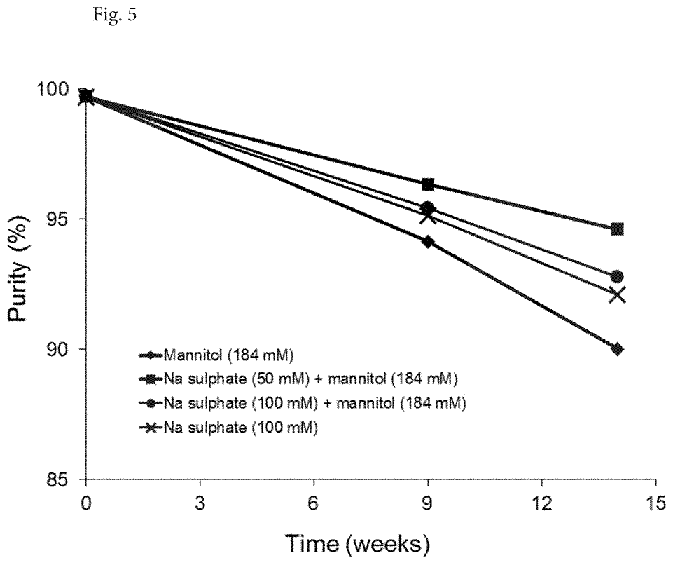

It was shown (Table 2 and FIG. 3) that the presence of citrate anion (from sodium citrate) led to a considerable improvement in stability of liraglutide. The effect appeared to be strongest at 50 mM citrate, but was also very apparent at citrate concentrations of 10 mM and 100 mM. At 100 mM citrate concentration, the effect appeared to be independent of the presence of mannitol. Similarly, the presence of higher concentration of phosphate anion (from sodium phosphate) had a stabilizing effect on liraglutide (FIG. 4). Whilst the control formulations (i.e. the first two formulations in Table 2) also contained a small amount of phosphate anion as a buffer, the stability was improved by increasing the concentration to either 50 mM or 100 mM. The presence of sulphate anion (from sodium sulphate) also improved the stability of liraglutide, particularly at 50 mM concentration (FIG. 5). The stabilizing effect of 100 mM sulphate anion was markedly less strong than that of 50 mM sulphate anion, but it was still measurable versus the multivalent anion free compositions.

TABLE 2
Purity of liraglutide assessed by visual assessment following storage
at 30° C. for 9 and 14 weeks. All formulations were adjusted to
pH 8.15 and contained liraglutide (6 mg/ml), sodium phosphate (8 mM),
phenol (58.4 mM) and additive(s) specified in the Table.
Purity was assessed by RP-HPLC.

| | MVA** | Purity (assessed as % RP- | | |
|---|---|---|---|---|
| | conc. | HPLC main peak) | | |
| Additive | (mM) | 0 weeks | 9 weeks | 14 weeks |
| Propylene glycol (184 mM) | 7.3 | 99.75 | 94.05 | 90.91 |
| Mannitol (184 mM) | 7.3 | 99.70 | 94.15 | 90.02 |
| Sodium citrate (10 mM) + | 17.3 | 99.68 | 96.30 | 94.77 |
| Mannitol (184 mM) | | | | |
| Sodium citrate (50 mM) + | 57.3 | 99.75 | 96.83 | 95.66 |
| Mannitol (184 mM) | | | | |
| Sodium citrate (100 mM) + | 107.3 | 99.75 | 96.37 | 94.86 |
| Mannitol (184 mM) | | | | |
| Sodium citrate (100 mM) | 107.3 | 99.74 | 95.89 | 94.26 |
| Phosphate (42 mM)* + | 45.6 | 99.83 | 96.01 | 93.34 |
| Mannitol (184 mM) | | | | |
| Phosphate (92 mM)* + | 91.2 | 99.72 | 96.02 | 93.52 |
| Mannitol (184 mM) | | | | |
| Phosphate (92 mM)* | 91.2 | 99.76 | 96.12 | 93.43 |
| Sodium sulphate (50 mM) + | 57.3 | 99.71 | 96.33 | 94.61 |
| Mannitol (184 mM) | | | | |
| Sodium sulphate (100 mM) + | 107.3 | 99.70 | 95.42 | 92.77 |
| Mannitol (184 mM) | | | | |
| Sodium sulphate (100 mM) | 107.3 | 99.68 | 95.12 | 92.11 |

*This concentration does not include 8 mM phosphate that was added to all formulations.
**Multivalent anion having a charge of at least minus 2 (based on pKₐ values of phosphoric acid of 2.2, 7.2 and 12.3 (25° C.), pKₐ values of citric acid of 3.1, 4.8 and 6.4 (25° C.) and pKₐ values of sulphuric acid of −3.0 and 2.0 (25° C.); pKₐ values obtained from CRC Handbook of Chemistry and Physics, 79^{th} Edition, 1998, D. R. Lide (phosphoric acid and sulphuric acid) and Merck Index (citric acid)).

Unless otherwise stated, pH, pKₐ and other physical parameters are determined at 25° C.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

SEQUENCE LISTING
SEQ ID NO: 1
(GLP-1(7-37)): HAEGTFTSDVSSYLEGQAAKEFIAWLVKGRG
(GLP-1(7-36)NH2):
SEQ ID NO: 2
HAEGTFTSDVSSYLEGQAAKEFIAWLVKGR-NH2
(exendin-4/Exexatide):
SEQ ID NO: 3
HGEGTFTSDLSKQMEEEAVRLFIEWLKNGGPSSGAPPPS-NH2
(exendin-3):
SEQ ID NO: 4
HSDGTFTSDLSKQMEEEAVRLFIEWLKNGGPSSGAPPPS-NH2
(Albiglutide):
SEQ ID NO: 5
HGEGTFTSDVSSYLEGQAAKEFIAWLVKGRHGEGTFTSDVSSYLEG QAA
KEFIAWLVKGRDAHKSEVAHRFKDLGEENFKALVLIAFAQYLQQCPFEDH
VKLVNEVTEFAKTCVADESAENCDKSLHTLFGDKLCTVATLRETYGEMAD
CCAKQEPERNECFLQHKDDNPNLPRLVRPEVDVMCTAFHDNEETFLKKYL
YEIARRHPYFYAPELLFFAKRYKAAFTECCQAADKAACLLPKLDELRDEG
KASSAKQRLKCASLQKFGERAFKAWAVARLSQRFPKAEFAEVSKLVTDLT
KVHTECCHGDLLECADDRADLAKYICENQDSISSKLKECCEKPLLEKSHC
IAEVENDEMPADLPSLAADFVESKDVCKNYAEAKDVFLGMFLYEYARRHP
DYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFS
ALEVDETYVPKEFNAETFTFHADICTLSEKERQIKKQTALVELVKHKPKA
TKEQLKAVMDDFAAFVEKCCKADDKETCFAEEGKKLVAASQAALGL
(Dulaglutide):
SEQ ID NO: 6
HGEGTFTSDVSSYLEEQAAKEFIAWLVKGGGGGGGSGGGGSGGGGSA ES
KYGPPCPPCPAPEAAGGPSVFLFPPKPKDTLMISRTPEVTCVVVDVS QE
DPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLG
(Liraglutide):
SEQ ID NO: 7
HAEGTFTSDVSSYLEGQAAK( [symbol] -E-palmitoyl)EFIAWLVRGRG
(Lixisenatide):
SEQ ID NO: 8
HGEGTFTSDLSKQMEEEAVRLFIEWLKNGGPSSGAPPSKKKKKK-NH2
(artificial sequence):
SEQ ID NO: 9
H-X¹-X²-G-T-F-T-S-D-X³-S-X⁴-X⁵-X⁶-E-X⁷-X⁸-A-X⁹-X¹⁰-
X¹¹-F-I-X¹²-W-L-X¹³-X¹⁴-G-X¹⁵
(artificial sequence):
SEQ ID NO: 10
PSSGAPPPS
(artificial sequence):
SEQ ID NO: 11
PSSGAPPSKKKKKK
(exemplary serum albumin sequence):
SEQ ID NO: 12
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL
(exemplary FcIgG4 sequence):
SEQ ID NO: 13
GGGGGSGGGGSGG GGSA ESKYGPPCPPCPAPEAAGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLG

1. An aqueous solution composition comprising a GLP-1 receptor agonist as an active ingredient and multivalent anions having a charge of at least minus 2 as stabilising agent, wherein the total concentration of multivalent anions in the composition having a charge of at least minus 2 is at least 15 mM.

2. An aqueous solution composition according to claim 1, wherein the GLP-1 receptor agonist is an insulinotropic analogue or derivative of GLP-1(7-37) (SEQ ID NO: 1) or an insulinotropic analogue or derivative of GLP-1(7-36)NH2 (SEQ ID NO: 2).

3. An aqueous solution composition according to claim 1, wherein the GLP-1 receptor agonist has or comprises the sequence set out below (SEQ ID NO: 9):
H-X¹-X²-G-T-F-T-S-D-X³-S-X⁴-X⁵-X⁶-E-X⁷-X⁸-A-X⁹-X¹⁰-X¹¹-F-I-X¹²-W-L-X¹³-X¹⁴-G-X¹⁵
wherein
X¹ is A, G or S; X² is E or D; X³ is V or L; X⁴ is S or K; X⁵ is Y or Q; X⁶ is L or M; X⁷ is G or E; X⁸ is Q or E; X⁹ is A or V; X¹⁰ is K or R; X¹¹ is E or L; X¹² is A or E; X¹³ is V or K; X¹⁴ is K, R or N;
and X¹⁵ is R or G;
or is a derivative thereof such as a derivative in which a simple amide (CONH₂) is formed of the C terminal COOH group and/or a side chain bears a lipophilic substituent, optionally via a linker.

4. An aqueous solution composition according to claim 1, wherein the GLP-1 receptor agonist is selected from the group consisting of albiglutide, dulaglutide, exenatide, liraglutide and lixisenatide.

5. An aqueous solution composition according to claim 4, wherein the GLP-1 receptor agonist is liraglutide or lixisenatide

6. An aqueous solution composition according to claim 5, wherein the GLP-1 receptor agonist is liraglutide.

7. An aqueous solution composition according to claim 1, wherein the concentration of GLP-1 receptor agonist in the composition is between 10 μg/mL and 50 mg/mL, such as between 200 μg/mL and 10 mg/mL, or between 1 mg/mL and 10 mg/mL.

8. An aqueous solution composition according to claim 1, wherein the multivalent anions as stabilizing agent have a charge of minus 2; or,
wherein the multivalent anions as stabilizing agent have a charge of minus 3; or
wherein the multivalent anions are a mixture of anions having charge of minus 2 and anions having charge of minus 3; or
wherein the multivalent anions having a charge of at least minus 2 are selected from divalent citrate anions, trivalent citrate anions, divalent sulphate anions, divalent phosphate anions, trivalent phosphate anions and mixtures thereof; or
wherein the multivalent anions are a mixture of at least two different multivalent anions having a charge of at least minus 2.

9.-12. (canceled)

13. An aqueous solution composition according to claim 8, wherein the multivalent anions having a charge of at least minus 2 are selected from divalent citrate anions, trivalent citrate anions and mixtures thereof.

14. An aqueous solution composition according to claim 1, wherein the total concentration of multivalent anions in the composition having a charge of at least minus 2 is at least 20 mM, such as at least 30 mM, at least 40 mM or at least 50 mM.

15. An aqueous solution composition according to claim 1, which additionally comprises a preservative such as a phenolic or benzylic preservative.

16. An aqueous solution composition according to claim 15, wherein the preservative is selected from the group consisting of phenol, m-cresol, chlorocresol, chlorophenol, benzyl alcohol, propyl paraben, methyl paraben, benzalkonium chloride and benzethonium chloride.

17. An aqueous solution composition according to claim 16, wherein the concentration of preservative is 10-100 mM, for example 20-80 mM, such as 25-50 mM.

18. An aqueous solution composition according to claim 1, further comprising a surfactant.

19. An aqueous solution composition according to claim 18, wherein the surfactant is a non-ionic surfactant such as a polysorbate.

20. An aqueous solution composition according to claim 18, wherein the surfactant is a cationic surfactant such as benzethonium salt or benzalkonium salt.

21. An aqueous solution composition according to claim 18, wherein the concentration of surfactant is 1-2000 μg/ml, e.g. 5-1000 μg/ml, e.g. 10-500 μg/ml, such as 10-200 μg/ml.

22. An aqueous solution composition according to claim 1, further comprising a tonicity modifier.

23. An aqueous solution composition according to claim 22, wherein the tonicity modifier is an uncharged tonicity modifier and is suitably selected from glycerol, mannitol, propylene glycol, trehalose, PEG300 and PEG400.

24. An aqueous solution composition according to claim 23, wherein the concentration of uncharged tonicity modifier is 50-1000 mM, for example 100-500 mM, such as about 300 mM.

25. An aqueous solution composition according to claim 22, wherein the tonicity modifier is a charged tonicity modifier and is suitably selected from sodium chloride, sodium sulphate, sodium acetate, sodium lactate, and amino acids such as glycine or arginine.

26. An aqueous solution composition according to claim 25, wherein the concentration of charged tonicity modifier is 25-500 mM, for example 50-250 mM such as about 150 mM.

27. An aqueous solution composition according to claim 1, wherein the composition does not contain an amino acid.

28. An aqueous solution composition according to claim 1, wherein the composition comprises an additional active ingredient, for example selected from glucagon, peptide YY, and a long acting insulin such as insulin glargine or insulin degludec.

29. An aqueous solution composition according to claim 1, which is a therapeutic composition.

30. (canceled)

31. A method of treating or preventing a disease or disorder caused by, associated with and/or accompanied by disturbances in carbohydrate and/or lipid metabolism comprising administering to a subject in need thereof a therapeutically effective amount of an aqueous solution composition according to claim 1.

32. The method according to claim 31, wherein the disease or disorder is selected from type 1 diabetes, type 2 diabetes, hyperglycaemia, impaired glucose tolerance, obesity and metabolic syndrome.

33. (canceled)

34. A method of improving the stability of an aqueous solution composition comprising a GLP-1 receptor agonist as active ingredient, which comprises adding multivalent anions having a charge of at least minus 2 to the composition.

35. A container containing one dose or a plurality of doses of an aqueous solution composition according to claim 1.

36. A container according to claim 35, which is a vial.

37. An injection device for single or multiple-use comprising a container according to claim 35 together with an injection needle.

38. An injection device according to claim 37, in the form of a pen.

39. An injection device according to claim 37, in the form of a pump.


SEQUENCE LISTING (13 sequences)

SEQ ID NO: 1 - protein, 31 aa, Homo sapiens
HAEGTFTSDVSSYLEGQAAKEFIAWLVKGRG

SEQ ID NO: 2 - protein, 30 aa, Homo sapiens
HAEGTFTSDVSSYLEGQAAKEFIAWLVKGR

SEQ ID NO: 3 - protein, 39 aa, artificial sequence
  exendin-4/Exexatide
HGEGTFTSDLSKQMEEEAVRLFIEWLKNGGPSSGAPPPS

SEQ ID NO: 4 - protein, 39 aa, artificial sequence
  exendin-3
HSDGTFTSDLSKQMEEEAVRLFIEWLKNGGPSSGAPPPS

SEQ ID NO: 5 - protein, 645 aa, artificial sequence
  Albiglutide
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 275 aa, artificial sequence
  Dulaglutide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 32 aa, artificial sequence
  Liraglutide
  MISC_FEATURE (21)..(21): Glu = gamma-Glu-palmitoyl
HAEGTFTSDVSSYLEGQAAKEEFIAWLVRGRG

SEQ ID NO: 8 - protein, 44 aa, artificial sequence
  Lixisenatide
HGEGTFTSDLSKQMEEEAVRLFIEWLKNGGPSSGAPPSKKKKKK

SEQ ID NO: 9 - protein, 30 aa, artificial sequence
  synthetic peptide
  VARIANT (2)..(2): Xaa is Ala, Gly or Ser
  VARIANT (3)..(3): Xaa is Glu or Asp
  VARIANT (10)..(10): Xaa is Val or Leu
  VARIANT (12)..(12): Xaa is Ser or Lys
  VARIANT (13)..(13): Xaa is Tyr or Gln
  VARIANT (14)..(14): Xaa is Leu or Met
  VARIANT (16)..(16): Xaa is Gly or Glu
  VARIANT (17)..(17): Xaa is Gln or Glu
  VARIANT (19)..(19): Xaa is Ala or Val
  VARIANT (20)..(20): Xaa is Lys or Arg
  VARIANT (21)..(21): Xaa is Glu or Leu
  VARIANT (24)..(24): Xaa is Ala or Glu
  VARIANT (27)..(27): Xaa is Val or Lys
  VARIANT (28)..(28): Xaa is Lys, Arg or Asn
  VARIANT (30)..(30): Xaa is Arg or Gly
HXXGTFTSDXSXXXEXXAXXXFIXWLXXGX

SEQ ID NO: 10 - protein, 9 aa, artificial sequence
  synthetic peptide
PSSGAPPPS

SEQ ID NO: 11 - protein, 14 aa, artificial sequence
  synthetic peptide
PSSGAPPSKKKKKK

SEQ ID NO: 12 - protein, 585 aa, Homo sapiens [sequence omitted]

SEQ ID NO: 13 - protein, 245 aa, Homo sapiens
GGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG